(19) United States
(12) Patent Application Publication
(10) Pub. No.: US 2010/0247574 A1
(43) Pub. Date: Sep. 30, 2010

(54) CHIMERIC NEWCASTLE DISEASE VIRUS VLPs

(76) Inventors: Kutub Mahmood, Cupertino, CA (US); Gale Smith, Gaithersburg, MD (US); Peter Pushko, Frederick, MD (US)

Correspondence Address: COOLEY LLP;ATTN: Patent Group, Suite 1100, 777 - 6th Street, NW, WASHINGTON, DC, 20001, US

(21) Appl. No.: 12/527,844

(22) PCT Filed: Feb. 21, 2008
(86) PCT No.: PCT/US08/54570

§ 371 Date: Jun. 9, 2010

Related U.S. Application Data
(60) Provisional application No. 60/902,337, filed on Feb. 21, 2007.

(51) Int. Cl. A61K 39/17; C07K 19/00; A61P 31/14; C12P 21/06

(52) U.S. Cl. 424/214.1; 530/403; 435/69.1

(57) ABSTRACT

The present invention discloses and claims chimeric virus like particles (VLPs) that express and/or contains Newcastle disease matrix protein. The invention includes vector constructs comprising said proteins, cells comprising said constructs, formulations and vaccines comprising chimeric VLPs of the inventions. The invention also includes methods of making and administrating chimeric VLPs to vertebrates, including methods of inducing immunity to infections.

This application claim priority to U.S. application 60/902,337, filed Feb. 21, 2007, which is incorporated by reference herein in its entirety for all purposes.

### BACKGROUND

Vaccination is based on a simple principle of immunity: once exposed to an infectious agent, an animal mounts an immune defense that provides lifelong protection against disease caused by the same agent. The goal of vaccination is to induce an animal to mount the defense prior to infection. Conventionally, this has been accomplished through the use of live attenuated or whole inactivated forms of the infectious agents as immunogens. The success of these approaches depends on the presentation of native antigen which elicits the complete range of immune responses obtained in natural infections.

Despite their considerable success, conventional vaccine methodologies are subject to a number of potential limitations. Insufficiently inactivated vaccines may cause the disease they are designed to prevent. Attenuated strains can mutate to become more virulent or non-immunogenic. In addition, viruses that can establish latency, such as the herpesviruses, are of particular concern, as it is not known whether there are any long-term negative consequences of latent infection by attenuated strains. Finally, there are no efficient means of growing many types of viruses.

Advances in recombinant DNA technology offer the potential for developing vaccines based on the use of defined antigens as immunogens, rather than the intact infectious agent. These include peptide vaccines, consisting of chemically synthesized, immunoreactive epitopes; subunit vaccines, produced by expression of viral proteins in recombinant heterologous cells; and the use of live viral vectors for the presentation of one or more defined antigens.

Both peptide and subunit vaccines are subject to a number of potential limitations. A major problem is the difficulty of ensuring that the conformation of the engineered proteins mimics that of the antigens in their natural environment. Suitable adjuvants and, in the case of peptides, carrier proteins, must be used to boost the immune response. In addition these vaccines elicit primarily humoral responses, and thus may fail to evoke effective immunity. Subunit vaccines are often ineffective for diseases in which whole inactivated virus can be demonstrated to provide protection.

Virus-like particles (VLPs) closely resemble mature virions, but they do not contain viral genomic material (e.g., viral genomic RNA). Therefore, VLPs are nonreplicative in nature, which make them safe for administration in the form of an immunogenic composition (e.g., vaccine). In addition, VLPs can be engineered to express viral envelope glycoproteins on the surface of the VLP, which is their most native physiological configuration. Moreover, since VLPs resemble intact virions and are multivalent particulate structures, VLPs may be more effective in inducing neutralizing antibodies to the envelope glycoprotein than soluble envelope protein antigens. Further, VLPs can be administered safely and repeatedly to vaccinated hosts, unlike many recombinant vaccine approaches.

The matrix-like proteins of many enveloped RNA viruses play a pivotal role in virus assembly and release (Pornillos et al. (2002) Trends Cell Biol., 12, 569-579). These proteins are often sufficient for release of particles. For example, expression of retroviral Gag precursor protein, in the absence of other viral components, results in the assembly and release of Gag virus-like particles (Delchambre et al. (1989) EMBO J. 8, 2653-2660). Matrix proteins from Ebola virus, vesicular stomatitis virus, and influenza virus, when expressed alone are released as VLPs (Jasenosky et al. (2004) Virus Res. 106, 181-188; Jayakar et al. Virus Res. 106, 117-132; Gomez-Puertas et al. J. Virol. 74, 11538-11547). The human parainfluenza virus type 1 (PIV1) and the Sendai virus (SV) M proteins expressed alone induces release of VLPs (Coronel et al. (1999) J. Virol. 73, 7035-7038; Sakaguchi et al. Virology 263, 230-243). Expression of M protein is also required for simian virus 5 VLP (SV5) formation (Schmitt et al. (2002) J. Virol. 76, 3952-3964), although other proteins may also be required.

Newcastle disease virus M protein when expressed in a host cell, induces formation and release of VLPs (Pantua et al. (2006) J. Virol., 80, 11062-11073). The inventors have taken advantage of the property of NDV M protein and have devised novel VLPs, antigenic formulations and vaccines to help prevent, treat, manage and/or ameliorate infectious diseases in vertebrates.

### SUMMARY OF THE INVENTION

The present invention comprises a chimeric virus like particle (VLP) comprising a Newcastle Disease Virus (NDV) core protein (M) and at least one protein from a different infectious agent. In one embodiment, said protein from an infectious agent is a viral protein.

In another embodiment, said viral protein is an envelope associated protein. In another embodiment, envelope associated protein is expressed on the surface of the VLP. In another embodiment, said VLP comprises a chimeric protein wherein said chimeric protein comprises said protein from a different infectious agent fused to a parainfluenza virus (PIV) protein. In another embodiment, said VLP comprises a chimeric protein, wherein said chimeric protein comprises said viral protein fused to a NDV protein.

The present invention also comprises, a method of producing a chimeric VLP, comprising transfecting vectors encoding a Newcastle Disease Virus (NDV) core protein (M) and at least one protein from a different infectious agent and expressing said vectors under conditions that allow VLPs to be formed. In one embodiment, said protein from an infectious agent is a viral protein. In another embodiment, said viral protein is from a virus selected the group consisting of influenza virus, dengue virus, yellow virus, Herpes simplex virus I and II, rabies virus, parainfluenza virus, varicella zoster virus, respiratory syncytial virus, rabies virus, human immunodeficiency virus, corona virus and hepatitis virus.

The present invention also comprises, an antigenic formulation comprising a chimeric VLP comprising a Newcastle Disease Virus (NDV) core protein (M) and at least one protein from a different infectious agent. In one embodiment, said viral protein is expressed on the surface of the VLP. In another embodiment, said viral protein comprises an epitope that will generate a protective immune response in a vertebrate. In another embodiment, said portion of the viral protein comprises an epitope that will generate a protective immune response in a vertebrate. In another embodiment, said antigenic formulation comprises an adjuvant. In another embodiment, said adjuvant are Novasomes.

The present invention also comprises, a vaccine comprising a chimeric VLP comprising a Newcastle Disease Virus (NDV) core protein (M) and at least one protein from a different infectious agent. In one embodiment, protein from an infectious agent is a viral protein. In another embodiment, said viral protein comprises an epitope that will generate a protective immune response in a vertebrate. In another embodiment, said vaccine comprises an adjuvant. In another embodiment, said adjuvant are Novasomes. In another embodiment, said VLPs are blended together to create a multivalent formulation.

The present invention also comprises, a method of inducing immunity in a vertebrate comprising administering to said vertebrate chimeric VLPs comprising a Newcastle Disease Virus (NDV) core protein (M) and at least one viral protein from a different virus. In one embodiment, said protein from an infectious agent is a viral protein. In another embodiment, wherein said immune response is a humoral immune response. In another embodiment, said immune response is a cellular immune response.

### BRIEF DESCRIPTION OF THE DRAWINGS

FIG. 1. represents constructs for making chimeric NDV VLPs comprising influenza proteins.

### DETAILED DESCRIPTION

### Definitions

As used herein the term “adjuvant” refers to a compound that, when used in combination with a specific immunogen (e.g. a VLP) in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses.

As used herein an “effective dose” generally refers to that amount of VLPs of the invention sufficient to induce immunity, to prevent and/or ameliorate an infection or to reduce at least one symptom of an infection and/or to enhance the efficacy of another dose of a VLP. An effective dose may refer to the amount of VLPs sufficient to delay or minimize the onset of an infection. An effective dose may also refer to the amount of VLPs that provides a therapeutic benefit in the treatment or management of an infection. Further, an effective dose is the amount with respect to VLPs of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

As used herein, the term “effective amount” refers to an amount of VLPs necessary or sufficient to realize a desired biologic effect. An effective amount of the composition would be the amount that achieves a selected result, and such an amount could be determined as a matter of routine by a person skilled in the art. For example, an effective amount for preventing, treating and/or ameliorating an infection could be that amount necessary to cause activation of the immune system, resulting in the development of an antigen specific immune response upon exposure to VLPs of the invention. The term is also synonymous with “sufficient amount.”

As used herein, the term “multivalent” refers to VLPs which have multiple antigenic proteins against multiple types or strains of agents.

As used herein the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interleukins (e.g., IL-1, IL-2, IL-3, IL-4, IL-6, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, CD28 etc. The immune stimulator molecules can be administered in the same formulation as VLPs of the invention, or can be administered separately. Either the protein or an expression vector encoding the protein can be administered to produce an immunostimulatory effect.

As used herein the term “protective immune response” or “protective response” refers to an immune response mediated by antibodies against an infectious agent, which is exhibited by a vertebrate (e.g., a human), that prevents or ameliorates an infection or reduces at least one symptom thereof. VLPs of the invention can stimulate the production of antibodies that, for example, neutralize infectious agents, blocks infectious agents from entering cells, blocks replication of said infectious agents, and/or protect host cells from infection and destruction. The term can also refer to an immune response that is mediated by T-lymphocytes and/or other white blood cells against an infectious agent, exhibited by a vertebrate (e.g., a human), that prevents or ameliorates RSV infection or reduces at least one symptom thereof.

As use herein, the term “infectious agent” refers to microorganisms that cause an infection in a vertebrate. Usually, the organisms are viruses, bacteria, parasites and/or fungi.

As use herein, the term “antigenic formulation” or “antigenic composition” refers to a preparation which, when administered to a vertebrate, e.g. a mammal, will induce an immune response.

As used herein, the term “vaccine” refers to a formulation which contains VLPs of the present invention, which is in a form that is capable of being administered to a vertebrate and which induces a protective immune response sufficient to induce immunity to prevent and/or ameliorate an infection and/or to reduce at least one symptom of an infection and/or to enhance the efficacy of another dose of VLPs. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

As use herein, the term “vertebrate” or “subject” or “patient” refers to any member of the subphylum cordata, including, without limitation, humans and other primates, including non-human primates such as chimpanzees and other apes and monkey species. Farm animals such as cattle, sheep, pigs, goats and horses; domestic mammals such as dogs and cats; laboratory animals including rodents such as mice, rats (including cotton rats) and guinea pigs; birds, including domestic, wild and game birds such as chickens, turkeys and other gallinaceous birds, ducks, geese, and the like are also non-limiting examples. The terms “mammals” and “animals” are included in this definition. Both adult and newborn individuals are intended to be covered. In particular, infants and young children are appropriate subjects or patients for a RSV vaccine.

As used herein, the term “virus-like particle” (VLP) refers to a structure that in at least one attribute resembles a virus but which has not been demonstrated to be infectious. Virus-like particle in accordance with the invention do not carry genetic information encoding for the proteins of virus-like particles. In general, virus-like particles lack a viral genome and, therefore, are noninfectious. In addition, virus-like particles can often be produced in large quantities by heterologous expression and can be easily purified.

A used herein, the term “chimeric VLP” refers to VLPs that contain proteins or portions of proteins from at least two different agents. Usually, one of the proteins is a derived from a virus that can drive the formation of VLPs from host cells. Examples, for illustrative purposes, are Newcastle M and/or influenza M protein. The terms Newcastle VLPs and chimeric VLPs can be used interchangeably where appropriate.

As used herein, the terms “NDV matrix,” “NDV M” or “NDV core” protein refer to a NDV membrane protein that, when expressed in a host cell, induces formation of enveloped VLPs. A representative NDV M protein is SEQ ID No. 1. The terms also comprises any variants, derivatives and/or fragments of NDV M that, when expressed in a host cell, induces formation of VLPs. The term also encompasses nucleotide sequences which encode for NDV M and/or any variants, derivatives and/or fragments thereof that when transfected (or infected) into a host cell will express NDV M protein and induce formation of VLPs.

### VLPs of the Invention and Methods of Making VLPs

In general, virus-like particles lack a viral genome and, therefore, are noninfectious. In addition, virus-like particles can often be produced in large quantities by heterologous expression and can be easily purified. Virus-like particles (“VLPs”) comprises at least a viral core protein. This core protein will drive budding and release of particles from a host cell. Examples of such proteins comprise RSV M, influenza M1, HIV gag, and vesicular stomatis virus (VSV) M protein. Recently, it has been shown that when the M protein of Newcastle disease virus (NDV) is expressed in host cells, particles are formed and released (Pantua et al. (2006) J. Virol., 80, 11062-11073). However, useful VLPs as immunogens typically will need a least one protein on the surface of the VLP. These VLPs would be useful for inducing an immune response against the protein or for targeting VLPs to specific cells. Although VLPs comprising a core protein and protein from the same virus are useful, this type of VLP would be limited to vaccines and other uses specific to the virus. It would be useful to have a platform in which VLPs can be made with proteins on the surface of the VLPs from different agents. For the purposes of this invention, such VLPs are referred to as “chimeric VLPs.” These VLPs would be useful for, among other things, for designing vaccines against diseases caused by different agents.

Thus, the invention comprises a chimeric virus like particle (VLP) comprising a Newcastle Disease Virus (NDV) core protein (M) and at least one protein from a different infectious agent. In one embodiment, said protein from an infectious agent is a viral protein. In another embodiment, said viral protein is an envelope associated protein. In another embodiment, said envelope associated protein is expressed on the surface of the VLP. In another embodiment, said envelope associated protein comprises an epitope that will generate a protective immune response in a vertebrate.

VLPs of the invention are useful for preparing vaccines and immunogenic compositions. One important feature of VLPs is the ability to present surface proteins so that the immune system of a vertebrate induces an immune response against said protein. However, not all proteins can be expressed or presented on the surface of VLPs. There may be many reasons why certain proteins are not expressed or presented, or be poorly expressed or presented, on the surface of the VLPs. One reason is that said protein is not directed to the membrane of a host cell or that said protein does not have a transmembrane domain. However, viruses do have the natural ability to express certain proteins on the surface of their structures.

Thus, one embodiment the invention comprises VLPs which comprise a chimeric protein wherein said chimeric protein comprises a protein from an infectious agent fused to a NDV or parainfluenza virus (PIV) protein. PIV is related to NDV. Thus, PIV components can be readily directed to the surface of the VLP, as NDV proteins are directed. Constructing chimeric protein with PIV or NDV virus proteins, such as fusion (F) or hemeagglutinin (HN) or fragments thereof, is advantageous because said chimeric proteins can direct the cell machinery into incorporating said chimeric proteins into the VLP. In another embodiment, said PIV protein is selected from the group consisting of PIV HN and F proteins or fragments thereof. In another embodiment, said protein from an infectious agent is a viral protein. In another embodiment, said chimeric protein comprises a portion of said viral protein and a portion of said PIV protein. In another embodiment, said portion of the viral protein is expressed on the surface of the VLP. In another embodiment, said portion of the viral protein comprises an epitope that will generate a protective immune response in a vertebrate. In another embodiment, said portion of the PIV protein associates, directly or indirectly, with the NDV M protein.

In another embodiment, the invention comprises chimeric VLPs that comprise a chimeric protein wherein said chimeric protein comprises a protein from an infectious agent fused to a NDV protein. In another embodiment, said protein from an infectious agent is a viral protein. In another embodiment, said NDV protein is selected from the group consisting of NP, F, and HN proteins. In another embodiment, said chimeric protein comprises a portion of said viral protein and a portion of said NDV protein. In another embodiment, said portion of the viral protein is expressed on the surface of the VLP. In another embodiment, said portion of the viral protein comprises an epitope that will generate a protective antibody response in a vertebrate. In another embodiment, said portion of the NDV protein associates, directly or indirectly, with the NDV M protein. In another embodiment, said chimeric NDV VLPs comprises a chimeric protein with the transmembrane and/or C-terminal domain of NDV HN and/or F protein fused to the external domains of proteins of an infection agent, such as influenza, VZV, RSV and/or Dengue virus. In another embodiment, said chimeric NDV VLPs comprise a chimeric protein comprising the external domains of influenza HA and/or NA protein and the transmembrane and/or C-terminal domain NDV HN and/or F proteins (see SEQ ID NO 10 for an example). In another embodiment, said chimeric VLP comprises SEQ ID NO 10.

Infectious agents can be viruses, bacteria and/or parasites. A protein that may be expressed on the surface of chimeric NDV VLPs can be derived from viruses, bacteria and/or parasites. The proteins derived from viruses, bacteria and/or parasites can induce an immune response (cellular and/or humoral) in a vertebrate that will prevent, treat, manage and/or ameliorate an infectious disease in said vertebrate.

Non-limiting examples of viruses from which said infectious agent proteins can be derived from are the following: seasonal, avian or pandemic influenza (A and B, e.g. HA and/or NA), coronavirus (e.g. SARS), hepatitis viruses A, B, C, D & E3, human immunodeficiency virus (HIV), herpes viruses 1, 2, 6 & 7, cytomegalovirus, varicella zoster, papilloma virus, Epstein Barr virus, parainfluenza viruses, adenoviruses, bunya viruses (e.g. hanta virus), coxsakie viruses, picoma viruses, rotaviruses, rhinoviruses, rubella virus, mumps virus, measles virus, Rubella virus, polio virus (multiple types), adeno virus (multiple types), parainfluenza virus (multiple types), avian influenza (various types), shipping fever virus, Western and Eastern equine encephalomyelitis, Japanese encephalomyelitis, fowl pox, rabies virus, slow brain viruses, rous sarcoma virus, Papovaviridae, Parvoviridae, Picornaviridae, Poxviridae (such as Smallpox or Vaccinia), Reoviridae (e.g., Rotavirus), Retroviridae (HTLV-I, HTLV-II, Lentivirus), Togaviridae (e.g., Rubivirus), respiratory syncytial virus (RSV), West Nile fever virus, Tick borne encephalitis, yellow fever, chikungunya virus, and dengue virus (all serotypes).

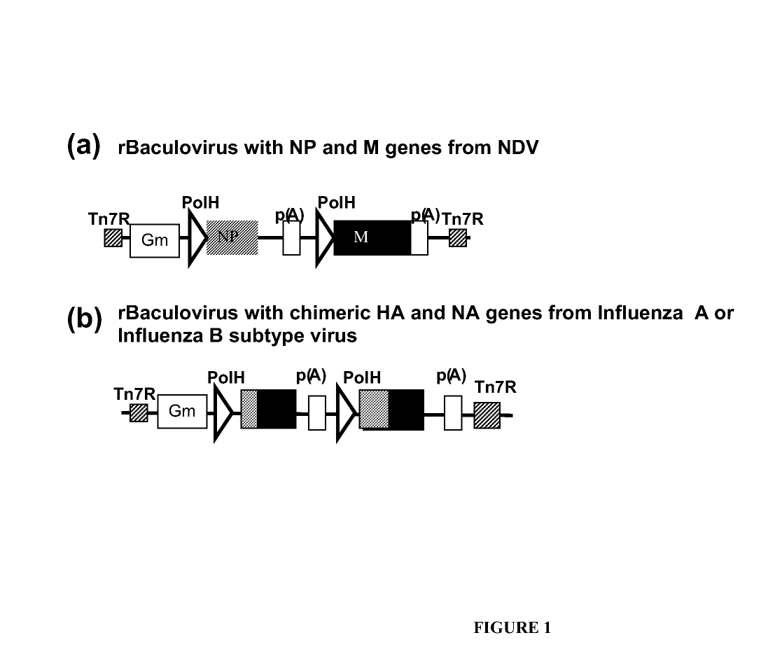

In another embodiment, the specific proteins from viruses may comprise: F and/or G protein from RSV, HA and/or NA from influenza virus (including avian or pandemic), S protein from coronavirus, gp160, gp140 and/or gp41 from HIV, gp I to IV and Vp from varicella zoster, E and preM/M from yellow fever virus, Dengue virus (all serotypes) or any flavivirus. Also included are any protein from a virus that can induce an immune response (cellular and/or humoral) in a vertebrate that can prevent, treat, manage and/or ameliorate an infectious disease in said vertebrate. An example of the above construct is illustrated in FIG. 1.

Non-limiting examples of bacteria from which said infectious agent proteins can be derived from are the following: B. pertussis, Leptospira pomona, S. paratyphi A and B, C. diphtheriae, C. tetani, C. botulinum, C. perfringens, C. feseri and other gas gangrene bacteria, B. anthracis, P. pestis, P. multocida, Neisseria meningitidis, N. gonorrheae, Hemophilus influenzae, Actinomyces (e.g., Norcardia), Acinetobacter, Bacillaceae (e.g., Bacillus anthrasis), Bacteroides (e.g., Bacteroides fragilis), Blastomycosis, Bordetella, Borrelia (e.g., Borrelia burgdorferi), Brucella, Campylobacter, Chlamydia, Coccidioides, Corynebacterium (e.g., Corynebacterium diptheriae), E. coli (e.g., Enterotoxigenic E. coli and Enterohemorrhagic E. coli), Enterobacter (e.g. Enterobacter aerogenes), Enterobacteriaceae (Klebsiella, Salmonella (e.g., Salmonella typhi, Salmonella enteritidis, Serratia, Yersinia, Shigella), Erysipelothrix, Haemophilus (e.g., Haemophilus influenza type B), Helicobacter, Legionella (e.g., Legionella pneumophila), Leptospira, Listeria (e.g., Listeria monocytogenes), Mycoplasma, Mycobacterium (e.g., Mycobacterium leprae and Mycobacterium tuberculosis), Vibrio (e.g., Vibrio cholerae), Pasteurellacea, Proteus, Pseudomonas (e.g., Pseudomonas aeruginosa), Rickettsiaceae, Spirochetes (e.g., Treponema spp., Leptospira spp., Borrelia spp.), Shigella spp., Meningiococcus, Pneumococcus and Streptococcus (e.g., Streptococcus pneumoniae and Groups A, B, and C Streptococci), Ureaplasmas, Treponema pollidum, Staphylococcus aureus, Pasteurella haemolytica, Corynebacterium diptheriae toxoid, Meningococcal polysaccharide, Bordetella pertusis, Streptococcus pneumoniae, Clostridium tetani toxoid, and Mycobacterium bovis.

Non-limiting examples of parasites from which said infectious agent proteins can be derived from are the following: leishmaniasis (Leishmania tropica mexicana, Leishmania tropica, Leishmania major, Leishmania aethiopica, Leishmania braziliensis, Leishmania donovani, Leishmania infantum, Leishmania chagasi), trypanosomiasis (Trypanosoma brucei gambiense, Trypanosoma brucei rhodesiense), toxoplasmosis (Toxoplasma gondii), schistosomiasis (Schistosoma haematobium, Schistosoma japonicum, Schistosoma mansoni, Schistosoma mekongi, Schistosoma intercalatum), malaria (Plasmodium virax, Plasmodium falciparium, Plasmodium malariae and Plasmodium ovale) Amebiasis (Entamoeba histolytica), Babesiosis (Babesiosis microti), Cryptosporidiosis (Cryptosporidium parvum), Dientamoebiasis (Dientamoeba fragilis), Giardiasis (Giardia lamblia), Helminthiasis and Trichomonas (Trichomonas vaginalis). The above lists are meant to be illustrative and by no means are meant to limit the invention to those particular bacterial, viral or parasitic organisms.

The invention also encompasses variants of the said proteins expressed on or in the VLPs of the invention. The variants may contain alterations in the amino acid sequences of the constituent proteins. The term “variant” with respect to a protein refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software.

Natural variants can occur due to mutations in the proteins. These mutations may lead to antigenic variability within individual groups of infectious agents, for example influenza. Thus, a person infected with an influenza strain develops antibody against that virus, as newer virus strains appear, the antibodies against the older strains no longer recognize the newer virus and reinfection can occur. The invention encompasses all antigenic and genetic variability of proteins from infectious agents for making VLPs.

General texts which describe molecular biological techniques, which are applicable to the present invention, such as cloning, mutation, cell culture and the like, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (“Ausubel”). These texts describe mutagenesis, the use of vectors, promoters and many other relevant topics related to, e.g., the cloning and mutating F and/or G molecules of RSV, etc. Thus, the invention also encompasses using known methods of protein engineering and recombinant DNA technology to improve or alter the characteristics of the proteins expressed on or in the VLPs of the invention. Various types of mutagenesis can be used to produce and/or isolate variant nucleic acids that encode for protein molecules and/or to further modify/mutate the proteins in or on the VLPs of the invention. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, is also included in the present invention. In one embodiment, mutagenesis can be guided by known information of the naturally occurring molecule or altered or mutated naturally occurring molecule, e.g., sequence, sequence comparisons, physical properties, crystal structure or the like.

The invention further comprises protein variants which show substantial biological activity, e.g., able to elicit an effective antibody response when expressed on or in VLPs of the invention. Such variants include deletions, insertions, inversions, repeats, and substitutions selected according to general rules known in the art so as have little effect on activity. An example of a mutation is to remove the cleavage site in a protein.

Methods of cloning said proteins are known in the art. For example, the gene encoding a specific Newcastle protein can be chemically synthesized as a synthetic gene or can be isolated by RT-PCR from polyadenylated mRNA extracted from cells which had been infected with the said virus. The resulting gene product can be cloned as a DNA insert into a vector. The term “vector” refers to the means by which a nucleic acid can be propagated and/or transferred between organisms, cells, or cellular components. Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. In many, but not all, common embodiments, the vectors of the present invention are plasmids or bacmids.

Thus, the invention comprises nucleotides that encode the proteins, including chimeric proteins, cloned into an expression vector that can be expressed in a cell that induces the formation of VLPs of the invention. An “expression vector” is a vector, such as a plasmid that is capable of promoting expression, as well as replication of a nucleic acid incorporated therein. Typically, the nucleic acid to be expressed is “operably linked” to a promoter and/or enhancer, and is subject to transcription regulatory control by the promoter and/or enhancer. In one embodiment, said nucleotides encode for a chimeric protein (e.g. PIV or NDV chimeric proteins as discussed above). In another embodiment, said vector comprises nucleotides that encode the NDV M protein and at least one protein from an infectious agent. In another embodiment, said vector comprises nucleotides that encode the NDV M protein and at least one protein from an infectious agent, or portions thereof, fused to PIV or NDV, or portions thereof. In another embodiment, the expression vector is a baculovirus vector.

In some embodiments, mutations containing alterations which produce silent substitutions, additions, or deletions, but do not alter the properties or activities of the encoded protein or how the proteins are made. Nucleotide variants can be produced for a variety of reasons, e.g., to optimize codon expression for a particular host (change codons those preferred by insect cells such as Sf9 cells, see SEQ ID NO 5, 6, 7 and 8). See U.S. patent publication 2005/0118191, herein incorporated by reference in its entirety for all purposes.

In addition, the nucleotides can be sequenced to ensure that the correct coding regions were cloned and do not contain any unwanted mutations. The nucleotides can be subcloned into an expression vector (e.g. baculovirus) for expression in any cell. The above is only one example of how the proteins for chimeric VLPs can be cloned. A person with skill in the art understands that additional methods are available and are possible.

The invention also provides for constructs and/or vectors that comprise nucleotides that encode for NDV structural genes, including, M, F, FIN and/or NP, or portions thereof, and/or PIV F and/or FIN, or portions thereof, and/or any chimeric protein described above. The vector may be, for example, a phage, plasmid, viral, or retroviral vector. The constructs and/or vectors that comprise the above constructs should be operatively linked to an appropriate promoter, such as the AcMNPV polyhedrin promoter (or other baculovirus), phage lambda PL promoter, the E. coli lac, phoA and tac promoters, the SV40 early and late promoters, and promoters of retroviral LTRs are non-limiting examples. Other suitable promoters will be known to the skilled artisan depending on the host cell and/or the rate of expression desired. The expression constructs will further contain sites for transcription initiation, termination, and, in the transcribed region, a ribosome-binding site for translation. The coding portion of the transcripts expressed by the constructs will preferably include a translation initiating codon at the beginning and a termination codon appropriately positioned at the end of the protein to be translated.

Expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Among vectors preferred are virus vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used with the invention comprise vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBac1 pWINEO, pSV2CAT, pOG44, pXT1 and pSG, pSVK3, pBPV, pMSG, and pSVL. Other suitable vectors will be readily apparent to the skilled artisan. In one embodiment, said vector that comprises NDV, M, F, FIN and/or NP, or portions thereof, and/or PIV F and/or FIN, or portions thereof, and/or any chimeric protein described above is pFastBac. In one embodiment, said vector that consists essentially of NDV, M, F, FIN and/or NP, or portions thereof, and/or PIV F and/or FIN, or portions thereof, and/or any chimeric protein described above is pFastBac. In one embodiment, said vector that consists of NDV, M, F, FIN and/or NP, or portions thereof, and/or PIV F and/or FIN, or portions thereof, and/or any chimeric protein described above is pFastBac.

Next, the recombinant constructs mentioned above could be used to transfect, infect, or transform and can express NDV M protein and NDV, F, FIN and/or NP, or portions thereof, and/or PIV F and/or FIN, or portions thereof, and/or any chimeric protein described above into eukaryotic cells and/or prokaryotic cells. Thus, the invention provides for host cells which comprise a vector (or vectors) that contain nucleic acids which the constructs described above in said host cell under conditions which allow the formation of VLPs.

Among eukaryotic host cells are yeast, insect, avian, plant, C. elegans (or nematode) and mammalian host cells. Non limiting examples of insect cells are, Spodoptera frugiperda (Sf) cells, e.g. Sf9, Sf21, Trichoplusia ni cells, e.g. High Five cells, and Drosophila S2 cells. Examples of fungi (including yeast) host cells are S. cerevisiae, Kluyveromyces lactis (K. lactis), species of Candida including C. albicans and C. glabrata, Aspergillus nidulans, Schizosaccharomyces pombe (S. pombe), Pichia pastoris, and Yarrowia lipolytica. Examples of mammalian cells are COS cells, baby hamster kidney cells, mouse L cells, LNCaP cells, Chinese hamster ovary (CHO) cells, human embryonic kidney (HEK) cells, and African green monkey cells, CV1 cells, HeLa cells, MDCK cells, Vero and Hep-2 cells. Xenopus laevis oocytes, or other cells of amphibian origin, may also be used. Prokaryotic host cells include bacterial cells, for example, E. coli, B. subtilis, and mycobacteria.

The present invention comprises a method of producing a chimeric VLP, comprising transfecting vectors encoding a Newcastle Disease Virus (NDV) core protein (M) and at least one viral protein from a different virus and expressing said vectors under conditions that allow VLPs to be formed. In another embodiment, said viral protein is an envelope associated protein. In another embodiment, said VLP comprises a chimeric protein wherein said chimeric protein comprises said viral protein fused to a parainfluenza virus (PIV) protein. In another embodiment, said PIV protein is selected from the group consisting of FIN and F proteins. In another embodiment, said chimeric protein comprises a portion of said viral protein and a portion of said PIV protein. In another embodiment, said portion of the PIV protein associates with the NDV M protein. In another embodiment, said VLP comprises a chimeric protein wherein said chimeric protein comprises said viral protein fused to a NDV protein. In another embodiment, said NDV protein is selected from the group consisting of NP, F, and FIN proteins. In another embodiment, said viral protein is from a virus selected the group consisting of influenza virus, dengue virus, yellow virus, Herpes simplex virus I and II, rabies virus, parainfluenza virus, varicella zoster virus, respiratory syncytial virus, rabies virus, human immunodeficiency virus, corona virus and hepatitis virus.

In another embodiment, said chimeric protein comprises a portion of said viral protein and a portion of said NDV protein. In another embodiment, said portion of the NDV protein associates with the NDV M protein. In another embodiment, said viral protein is from a virus selected the group consisting of influenza virus, dengue virus, yellow virus, Herpes simplex virus I and II, rabies virus, parainfluenza virus, varicella zoster virus, respiratory syncytial virus, rabies virus, human immunodeficiency virus, corona virus and hepatitis virus. In another embodiment, said influenza viral protein is HA and/or NA. In another embodiment, said respiratory syncytial virus viral protein is F and/or G. In another embodiment, said Dengue virus viral protein is E and/or preM/M. In another embodiment, said chimeric NDV VLPs comprises a chimeric protein with the transmembrane and/or C-terminal domain of NDV HN and/or F protein fused to the external domains of proteins of an infection agent, such as influenza, VZV, RSV and/or Dengue virus. In another embodiment, said chimeric NDV VLPs comprise a chimeric protein comprising the external domains of influenza HA and/or NA protein and the transmembrane and/or C-terminal domain NDV FIN and/or F proteins (see SEQ ID NO 10 for an example). In another embodiment, said chimeric VLP comprises SEQ ID NO 10.

Vectors, e.g., vectors comprising polynucleotides the above constructs, can be transfected into host cells according to methods well known in the art. For example, introducing nucleic acids into eukaryotic cells can be by calcium phosphate co-precipitation, electroporation, microinjection, lipofection, and transfection employing polyamine transfection reagents. In one embodiment, said vector is a recombinant baculovirus. In another embodiment, said recombinant baculovirus is transfected into a eukaryotic cell. In a preferred embodiment, said cell is an insect cell. In another embodiment, said insect cell is a Sf9 cell.

In another embodiment, said vector and/or host cell comprise nucleotides that encode NDV M protein and NDV F, HN and/or NP protein, or portions thereof, and/or PIV F and/or FIN proteins, or portions thereof, and/or any chimeric protein described above. In another embodiment, said vector and/or host cell consists essentially of NDV M protein and NDV F, FIN and/or NP proteins, or portions thereof, and/or PIV F and/or FIN proteins, or portions thereof, and/or any chimeric protein described above. In a further embodiment, said vector and/or host cell consists of NDV M protein and NDV F, FIN and/or NP proteins, or portions thereof, and/or PIV F and/or FIN proteins, or portions thereof, and/or any chimeric protein described above. These vector and/or host cell that contain the above constructs, may also contain additional cellular constituents such as cellular proteins, baculovirus proteins, lipids, carbohydrates etc., but do not contain additional NDV proteins (other than fragments of the above described constructs).

This invention also provides for constructs and methods that will increase the efficiency of VLP production. For example, the addition of leader sequences to the constructs described above can improve the efficiency of protein transporting within the cell. For example, a heterologous signal sequence can be fused to NDV M protein and NDV F, FIN and/or NP proteins, or portions thereof, and/or PIV F and/or FIN proteins, or portions thereof, and/or any chimeric protein described above. In one embodiment, the signal sequence can be derived from the gene of an insect cell. In another embodiment, the signal peptide is the chitinase signal sequence, which works efficiently in baculovirus expression systems.

Another method to increase efficiency of VLP production is to codon optimize the nucleotides that encode NDV M protein and NDV F, FIN and/or NP proteins, or portions thereof, and/or PIV F and/or FIN proteins, or portions thereof, and/or any chimeric protein described above for a specific cell type. For examples of codon optimizing nucleic acids for expression in Sf9 cell see SEQ ID NO 5, 6, 7 and 8 and U.S. patent publication 2005/0118191, herein incorporated by reference in its entirety for all purposes.

The invention also provides for methods of producing VLPs, said methods comprising expressing NDV M protein and NDV F, FIN and/or NP proteins, or portions thereof, and/or PIV F and/or FIN proteins, or portions thereof, and/or any chimeric protein described above under conditions that allow VLP formation. Depending on the expression system and host cell selected, the VLPs are produced by growing host cells transformed by an expression vector under conditions whereby the recombinant proteins are expressed and VLPs are formed. In one embodiment, the invention comprises a method of producing a VLP, comprising transfecting vectors encoding at least a NDV M protein into a suitable host cell and expressing said protein under conditions that allow VLP formation. In another embodiment, said VLP comprises the NDV M protein and NDV F, FIN and/or NP proteins, or portions thereof, and/or PIV F and/or FIN proteins, or portions thereof, and/or any chimeric protein described above. In another embodiment, said eukaryotic cell is selected from the group consisting of, yeast, insect, amphibian, avian or mammalian cells. The selection of the appropriate growth conditions is within the skill or a person with skill of one of ordinary skill in the art.

In another embodiment, the method comprises making VLPs comprising a NDV M protein and at least one protein from another infectious agent. In another embodiment, said protein from another infectious agent is a viral protein. In another embodiment, said protein from an infectious agent is an envelope-associated protein. In another embodiment, said protein from another infectious agent is expressed on the surface of VLPs. In another embodiment, said protein from an infectious agent comprises an epitope that will generate a protective immune response in a vertebrate. In another embodiment, said protein from another infectious agent can associated with NDV M protein.

Methods to grow cells engineered to produce VLPs of the invention include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, said bioreactor is a stainless steel chamber. In another embodiment, said bioreactor is a pre-sterilized plastic bag (e.g. Cellbag®, Wave Biotech, Bridgewater, N.J.). In other embodiment, said pre-sterilized plastic bags are about 50 L to 1000 L bags.

The VLPs are then isolated using methods that preserve the integrity thereof, such as by gradient centrifugation, e.g., cesium chloride, sucrose and iodixanol, as well as standard purification techniques including, e.g., ion exchange and gel filtration chromatography.

The following is an example of how VLPs of the invention can be made, isolated and purified. Usually VLPs are produced from recombinant cell lines engineered to create VLPs when said cells are grown in cell culture (see above). A person of skill in the art would understand that there are additional methods that can be utilized to make and purify VLPs of the invention, thus the invention is not limited to the method described.

Production of VLPs of the invention can start by seeding Sf9 cells (non-infected) into shaker flasks, allowing the cells to expand and scaling up as the cells grow and multiply (for example from a 125-ml flask to a 50 L Wave bag). The medium used to grow the cell is formulated for the appropriate cell line (preferably serum free media, e.g. insect medium ExCell-420, JRH). Next, said cells are infected with recombinant baculovirus at the most efficient multiplicity of infection (e.g. from about 1 to about 3 plaque forming units per cell). Once infection has occurred, the NDV M protein and NDV F, FIN and/or NP proteins, or portions thereof, and/or PIV F and/or FIN proteins, or portions thereof, and/or any chimeric protein described above, are expressed from the virus genome, self assemble into VLPs and are secreted from the cells approximately 24 to 72 hours post infection. Usually, infection is most efficient when the cells are in mid-log phase of growth (4-8×10⁶ cells/ml) and are at least about 90% viable.

VLPs of the invention can be harvested approximately 48 to 96 hours post infection, when the levels of VLPs in the cell culture medium are near the maximum but before extensive cell lysis. The Sf9 cell density and viability at the time of harvest can be about 0.5×10⁶ cells/ml to about 1.5×10⁶ cells/ml with at least 20% viability, as shown by dye exclusion assay. Next, the medium is removed and clarified. NaCl can be added to the medium to a concentration of about 0.4 to about 1.0 M, preferably to about 0.5 M, to avoid VLP aggregation. The removal of cell and cellular debris from the cell culture medium containing VLPs of the invention can be accomplished by tangential flow filtration (TFF) with a single use, pre-sterilized hollow fiber 0.5 or 1.00 μm filter cartridge or a similar device.

Next, VLPs in the clarified culture medium can be concentrated by ultrafiltration using a disposable, pre-sterilized 500,000 molecular weight cut off hollow fiber cartridge. The concentrated VLPs can be diafiltrated against 10 volumes pH 7.0 to 8.0 phosphate-buffered saline (PBS) containing 0.5 M NaCl to remove residual medium components.

The concentrated, diafiltered VLPs can be furthered purified on a 20% to 60% discontinuous sucrose gradient in pH 7.2 PBS buffer with 0.5 M NaCl by centrifugation at 6,500×g for 18 hours at about 4° C. to about 10° C. Usually VLPs will form a distinctive visible band between about 30% to about 40% sucrose or at the interface (in a 20% and 60% step gradient) that can be collected from the gradient and stored. This product can be diluted to comprise 200 mM of NaCl in preparation for the next step in the purification process. This product contains VLPs and may contain intact baculovirus particles.

Further purification of VLPs can be achieved by anion exchange chromatography, or 44% isopycnic sucrose cushion centrifugation. In anion exchange chromatography, the sample from the sucrose gradient (see above) is loaded into column containing a medium with an anion (e.g. Matrix Fractogel EMD TMAE) and eluded via a salt gradient (from about 0.2 M to about 1.0 M of NaCl) that can separate the VLP from other contaminates (e.g. baculovirus and DNA/RNA). In the sucrose cushion method, the sample comprising the VLPs is added to a 44% sucrose cushion and centrifuged for about 18 hours at 30,000 g. VLPs form a band at the top of 44% sucrose, while baculovirus precipitates at the bottom and other contaminating proteins stay in the 0% sucrose layer at the top. The VLP peak or band is collected.

The intact baculovirus can be inactivated, if desired. Inactivation can be accomplished by chemical methods, for example, formalin or β-propiolactone (BPL). Removal and/or inactivation of intact baculovirus can also be largely accomplished by using selective precipitation and chromatographic methods known in the art, as exemplified above. Methods of inactivation comprise incubating the sample containing the VLPs in 0.2% of BPL for 3 hours at about 25° C. to about 27° C. The baculovirus can also be inactivated by incubating the sample containing the VLPs at 0.05% BPL at 4° C. for 3 days, then at 37° C. for one hour.

After the inactivation/removal step, the product comprising VLPs can be run through another diafiltration step to remove any reagent from the inactivation step and/or any residual sucrose, and to place the VLPs into the desired buffer (e.g. PBS). The solution comprising VLPs can be sterilized by methods known in the art (e.g. sterile filtration) and stored in the refrigerator or freezer.

The above techniques can be practiced across a variety of scales. For example, T-flasks, shake-flasks, spinner bottles, up to industrial sized bioreactors. The bioreactors can comprise either a stainless steel tank or a pre-sterilized plastic bag (for example, the system sold by Wave Biotech, Bridgewater, N.J.). A person with skill in the art will know what is most desirable for their purposes.

Expansion and production of baculovirus expression vectors and infection of cells with recombinant baculovirus to produce chimeric NDV VLPs can be accomplished in insect cells, for example Sf9 insect cells as previously described. In one embodiment, the cells are SF9 infected with recombinant baculovirus engineered to produce chimeric NDV VLPs.

### Pharmaceuticals or Vaccine Formulations and Administration

The pharmaceutical compositions useful herein contain a pharmaceutically acceptable carrier, including any suitable diluent or excipient, which includes any pharmaceutical agent that does not itself induce the production of an immune response harmful to the vertebrate receiving the composition, and which may be administered without undue toxicity and a VLP of the invention. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful as a vaccine and/or antigenic compositions for inducing a protective immune response in a vertebrate.

One embodiment of the invention comprises an antigenic formulation comprising a chimeric VLP comprising a Newcastle Disease Virus (NDV) core protein (M) and at and at least one protein from a different infectious agent. In one embodiment, said protein from an infectious agent is a viral protein. In another embodiment, said viral protein is expressed on the surface of the VLP. In another embodiment, said viral protein comprises an epitope that will generate a protective antibody response in a vertebrate. In another embodiment, said VLP comprises a chimeric protein, wherein said chimeric protein comprises said viral protein fused to a parainfluenza virus (PIV) protein. In another embodiment, said PIV protein is selected from the group consisting of HN and F proteins. In another embodiment, said chimeric protein comprises a portion of said viral protein and a portion of said PIV protein. In another embodiment, said portion of the viral protein is expressed on the surface of the VLP. In another embodiment, said portion of the viral protein comprises an epitope that will generate a protective antibody response in a vertebrate. In another embodiment, said portion of the PIV protein associates with the NDV M protein. In another embodiment, said VLP comprises a chimeric protein, wherein said chimeric protein comprises said viral protein fused to a NDV protein. In another embodiment, said NDV protein is selected from the group consisting of NP, F, and FIN proteins. In another embodiment, said chimeric protein comprises a portion of said viral protein and a portion of said NDV protein. In another embodiment, said portion of the viral protein is expressed on the surface of the VLP. In another embodiment, said portion of the viral protein comprises an epitope that will generate a protective antibody response in a vertebrate. In another embodiment, said portion of the NDV protein associates with the NDV M protein.

Another embodiment of the invention comprises a vaccine comprising a chimeric VLP comprising a Newcastle Disease Virus (NDV) core protein (M) and at and at least one protein from a different infectious agent. In one embodiment, said protein from an infectious agent is a viral protein. In one embodiment, said viral protein is expressed on the surface of the VLP. In one embodiment, said viral protein comprises an epitope that will generate a protective antibody response in a vertebrate. In one embodiment, said VLP comprises a chimeric protein wherein said chimeric protein comprises said viral protein fused to a parainfluenza virus (PIV) protein. In one embodiment, said PIV protein is selected from the group consisting of FIN and F proteins. In one embodiment, said chimeric protein comprises a portion of said viral protein and a portion of said PIV protein. In one embodiment, said portion of the viral protein is expressed on the surface of the VLP. In one embodiment, said portion of the viral protein comprises an epitope that will generate a protective antibody response in a vertebrate. In one embodiment, said portion of the PIV protein associates with the NDV M protein. In another embodiment, said VLP comprises a chimeric protein, wherein said chimeric protein comprises said viral protein fused to a NDV protein. In one embodiment, said NDV protein is selected from the group consisting of NP, F, and FIN proteins. In one embodiment, said chimeric protein comprises a portion of said viral protein and a portion of said NDV protein. In one embodiment, said portion of the viral protein is expressed on the surface of the VLP. In one embodiment, said portion of the viral protein comprises an epitope that will generate a protective antibody response in a vertebrate. In one embodiment, said portion of the NDV protein associates with the NDV M protein.

One embodiment of the invention comprises an antigenic formulation comprising a chimeric VLP comprising the NDV M protein and at least one protein from a different infectious agent. In one embodiment, said protein from an infectious agent is a viral protein. In another embodiment, wherein said viral protein is selected from the group consisting of influenza virus, dengue virus, yellow virus, Herpes simplex virus I and II, rabies virus, parainfluenza virus, varicella zoster virus, respiratory syncytial virus, rabies virus, human immunodeficiency virus, corona virus and hepatitis virus. In another embodiment, said influenza viral protein is HA and/or NA. In another embodiment, said respiratory syncytial virus viral protein is F and/or G. In another embodiment, said Dengue virus viral protein is E and/or preM/M. In another embodiment, said chimeric NDV VLPs comprises a chimeric protein with the transmembrane and/or C-terminal domain of NDV HN and/or F protein fused to the external domains of proteins of an infection agent, such as influenza, VZV, RSV and/or Dengue virus. In another embodiment, said chimeric NDV VLPs comprise a chimeric protein comprising the external domains of influenza HA and/or NA protein and the transmembrane and/or C-terminal domain NDV HN and/or F proteins (see SEQ ID NO 10 for an example). In another embodiment, said chimeric VLP comprises SEQ ID NO 10.

Another embodiment of the invention comprises different chimeric VLPs are blended together to create a multivalent formulation. In another embodiment, said antigenic, vaccine and/or multivalent formulation is administered to a vertebrate orally, intradermally, intranasally, intramuscularly, intraperitoneally, intravenously or subcutaneously.

Said formulations of the invention comprise a formulation comprising a chimeric VLP comprising the NDV M protein and at least one protein from a different infectious agent. described above and a pharmaceutically acceptable carrier or excipient. Pharmaceutically acceptable carriers include but are not limited to saline, buffered saline, dextrose, water, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co. N.J. current edition). The formulation should suit the mode of administration. In a preferred embodiment, the formulation is suitable for administration to humans, preferably is sterile, non-particulate and/or non-pyrogenic.

The composition, if desired, can also contain minor amounts of wetting or emulsifying agents, or pH buffering agents. The composition can be a solid form, such as a lyophilized powder suitable for reconstitution, a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

The invention also provides for a pharmaceutical pack or kit comprising one or more containers filled with one or more of the ingredients of the vaccine formulations of the invention. In a preferred embodiment, the kit comprises two containers, one containing VLPs and the other containing an adjuvant. Associated with such container(s) can be a notice in the form prescribed by a governmental agency regulating the manufacture, use or sale of pharmaceuticals or biological products, which notice reflects approval by the agency of manufacture, use or sale for human administration.

The invention also provides that the VLP formulation be packaged in a hermetically sealed container such as an ampoule or sachette indicating the quantity of composition. In one embodiment, the VLP composition is supplied as a liquid, in another embodiment, as a dry sterilized lyophilized powder or water free concentrate in a hermetically sealed container and can be reconstituted, e.g., with water or saline to the appropriate concentration for administration to a subject.

In an alternative embodiment, the VLP composition is supplied in liquid form in a hermetically sealed container indicating the quantity and concentration of the VLP composition. Preferably, the liquid form of the VLP composition is supplied in a hermetically sealed container at least about 50 μg/ml, more preferably at least about 100 μg/ml, at least about 200 μg/ml, at least 500 μg/ml, or at least 1 mg/ml.

Generally, chimeric NDV VLPs of the invention are administered in an effective amount or quantity (as defined above) sufficient to stimulate an immune response against one or more infectious agents. Preferably, administration of the VLP of the invention elicits immunity against an infectious agent. Typically, the dose can be adjusted within this range based on, e.g., age, physical condition, body weight, sex, diet, time of administration, and other clinical factors. The prophylactic vaccine formulation is systemically administered, e.g., by subcutaneous or intramuscular injection using a needle and syringe, or a needle-less injection device. Alternatively, the vaccine formulation is administered intranasally, either by drops, large particle aerosol (greater than about 10 microns), or spray into the upper respiratory tract. While any of the above routes of delivery results in an immune response, intranasal administration confers the added benefit of eliciting mucosal immunity at the site of entry of many viruses, including RSV and influenza.

Thus, the invention also comprises a method of formulating a vaccine or antigenic composition that induces immunity to an infection or at least one symptom thereof to a mammal, comprising adding to said formulation an effective dose of chimeric NDV VLPs.

Methods of administering a composition comprising VLPs (vaccine and/or antigenic formulations) include, but are not limited to, parenteral administration (e.g., intradermal, intramuscular, intravenous and subcutaneous), epidural, and mucosal (e.g., intranasal and oral or pulmonary routes or by suppositories). In a specific embodiment, compositions of the present invention are administered intramuscularly, intravenously, subcutaneously, transdermally or intradermally. The compositions may be administered by any convenient route, for example by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents. In some embodiments, intranasal or other mucosal routes of administration of a composition comprising VLPs of the invention may induce an antibody or other immune response that is substantially higher than other routes of administration. In another embodiment, intranasal or other mucosal routes of administration of a composition comprising VLPs of the invention may induce an antibody or other immune response that will induce cross protection against other strains or organisms that cause infection. For example, a chimeric NDV VLP comprising influenza protein, when administered to a vertebrate, can induce cross protection against several influenza strains. Administration can be systemic or local.

In yet another embodiment, the vaccine and/or antigenic formulation is administered in such a manner as to target mucosal tissues in order to elicit an immune response at the site of immunization. For example, mucosal tissues such as gut associated lymphoid tissue (GALT) can be targeted for immunization by using oral administration of compositions which contain adjuvants with particular mucosal targeting properties. Additional mucosal tissues can also be targeted, such as nasopharyngeal lymphoid tissue (NALT) and bronchial-associated lymphoid tissue (BALT).

Vaccines and/or antigenic formulations of the invention may also be administered on a dosage schedule, for example, an initial administration of the vaccine composition with subsequent booster administrations. In particular embodiments, a second dose of the composition is administered anywhere from two weeks to one year, preferably from about 1, about 2, about 3, about 4, about 5 to about 6 months, after the initial administration. Additionally, a third dose may be administered after the second dose and from about three months to about two years, or even longer, preferably about 4, about 5, or about 6 months, or about 7 months to about one year after the initial administration. The third dose may be optionally administered when no or low levels of specific immunoglobulins are detected in the serum and/or urine or mucosal secretions of the subject after the second dose. In a preferred embodiment, a second dose is administered about one month after the first administration and a third dose is administered about six months after the first administration. In another embodiment, the second dose is administered about six months after the first administration. In another embodiment, said VLPs of the invention can be administered as part of a combination therapy. For example, VLPs of the invention can be formulated with other immunogenic compositions, antivirals and/or antibiotics.

The dosage of the pharmaceutical formulation can be determined readily by the skilled artisan, for example, by first identifying doses effective to elicit a prophylactic or therapeutic immune response, e.g., by measuring the serum titer of virus specific immunoglobulins or by measuring the inhibitory ratio of antibodies in serum samples, or urine samples, or mucosal secretions. Said dosages can be determined from animal studies. A non-limiting list of animals used to study the efficacy of vaccines include the guinea pig, hamster, ferrets, chinchilla, mouse and cotton rat. Most animals are not natural hosts to infectious agents but can still serve in studies of various aspects of the disease. For example, any of the above animals can be dosed with a vaccine candidate, e.g. VLPs of the invention, to partially characterize the immune response induced, and/or to determine if any neutralizing antibodies have been produced. For example, many studies have been conducted in the mouse model because mice are small size and their low cost allows researchers to conduct studies on a larger scale.

In addition, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems.

As also well known in the art, the immunogenicity of a particular composition can be enhanced by the use of non-specific stimulators of the immune response, known as adjuvants. Adjuvants have been used experimentally to promote a generalized increase in immunity against unknown antigens (e.g., U.S. Pat. No. 4,877,611). Immunization protocols have used adjuvants to stimulate responses for many years, and as such, adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. The inclusion of any adjuvant described in Vogel et al., “A Compendium of Vaccine Adjuvants and Excipients (2^{nd} Edition),” herein incorporated by reference in its entirety for all purposes, is envisioned within the scope of this invention.

Exemplary, adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MF-59, Novasomes®, MHC antigens may also be used.

In one embodiment of the invention, the adjuvant is a paucilamellar lipid vesicle having about two to ten bilayers arranged in the form of substantially spherical shells separated by aqueous layers surrounding a large amorphous central cavity free of lipid bilayers. Paucilamellar lipid vesicles may act to stimulate the immune response several ways, as non-specific stimulators, as carriers for the antigen, as carriers of additional adjuvants, and combinations thereof. Paucilamellar lipid vesicles act as non-specific immune stimulators when, for example, a vaccine is prepared by intermixing the antigen with the preformed vesicles such that the antigen remains extracellular to the vesicles. By encapsulating an antigen within the central cavity of the vesicle, the vesicle acts both as an immune stimulator and a carrier for the antigen. In another embodiment, the vesicles are primarily made of nonphospholipid vesicles. In other embodiment, the vesicles are Novasomes. Novasomes® are paucilamellar nonphospholipid vesicles ranging from about 100 nm to about 500 nm. They comprise Brij 72, cholesterol, oleic acid and squalene. Novasomes have been shown to be an effective adjuvant for influenza antigens (see, U.S. Pat. Nos. 5,629,021, 6,387,373, and 4,911,928, herein incorporated by reference in their entireties for all purposes).

Another method of inducing an immune response can be accomplished by formulating the VLPs of the invention with “immune stimulators.” These are the body's own chemical messengers (cytokines) to increase the immune system's response. Immune stimulators include, but not limited to, various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immunostimulatory molecules can be administered in the same formulation as the RSV VLPs, or can be administered separately. Either the protein or an expression vector encoding the protein can be administered to produce an immunostimulatory effect. Thus in one embodiment, the invention comprises antigentic and vaccine formulations comprising an adjuvant and/or an immune stimulator.

Thus, one embodiment of the invention comprises a formulation comprising a chimeric VLP comprising a Newcastle Disease Virus (NDV) core protein (M), at least one protein from an infectious agent and adjuvant and/or an immune stimulator. In another embodiment, said adjuvant are Novasomes. In another embodiment, said formulation is suitable for human administration. In another embodiment, the formulation is administered to a vertebrate orally, intradermally, intranasally, intramuscularly, intraperitoneally, intravenously or subcutaneously. In another embodiment, different chimeric VLPs are blended together to create a multivalent formulation.

While stimulation of immunity with a single dose is preferred, additional dosages can be administered, by the same or different route, to achieve the desired effect. In neonates and infants, for example, multiple administrations may be required to elicit sufficient levels of immunity. Administration can continue at intervals throughout childhood, as necessary to maintain sufficient levels of protection against infections. Similarly, adults who are particularly susceptible to repeated or serious infections, such as, for example, health care workers, day care workers, family members of young children, the elderly, and individuals with compromised cardiopulmonary function may require multiple immunizations to establish and/or maintain protective immune responses. Levels of induced immunity can be monitored, for example, by measuring amounts of neutralizing secretory and serum antibodies, and dosages adjusted or vaccinations repeated as necessary to elicit and maintain desired levels of protection.

### Methods of Stimulating an Immune Response

As mentioned above, the VLPs of the invention are useful for preparing compositions that stimulate an immune response that confers immunity or substantial immunity to infectious agents. Both mucosal and cellular immunity may contribute to immunity to infectious agents and disease. Antibodies secreted locally in the upper respiratory tract are a major factor in resistance to natural infection. Secretory immunoglobulin A (sIgA) is involved in protection of the upper respiratory tract and serum IgG in protection of the lower respiratory tract. The immune response induced by an infection protects against reinfection with the same virus or an antigenically similar viral strain. For example, influenza undergoes frequent and unpredictable changes; therefore, after natural infection, the effective period of protection provided by the host's immunity may only be a few years against the new strains of virus circulating in the community.

Chimeric NDV VLPs of the invention can induce substantial immunity in a vertebrate (e.g. a human) when administered to said vertebrate. The substantial immunity results from an immune response against VLPs of the invention that protects or ameliorates infection or at least reduces a symptom of infection in said vertebrate. In some instances, if the said vertebrate is infected, said infection will be asymptomatic. The response may be not a fully protective response. In this case, if said vertebrate is infected with an infectious agent, the vertebrate will experience reduced symptoms or a shorter duration of symptoms compared to a non-immunized vertebrate.

In one embodiment, the invention comprises a method of inducing substantial immunity to an infection, or at least one symptom thereof, in a subject, comprising administering at least one effective dose of chimeric NDV VLPs. In another embodiment, the invention comprises a method of vaccinating a mammal against RSV comprising administering to said mammal a protection-inducing amount of VLPs comprising chimeric NDV VLPs. In one embodiment, said method comprises administering VLPs comprising NDV M protein and NDV F, HN and/or NP protein, or portions thereof, and/or PIV F and/or FIN proteins, or portions thereof, and/or any chimeric protein described above.

In another embodiment, the invention comprises a method of inducing a protective antibody response to an infection or at least one symptom thereof in a subject, comprising administering at least one effective dose of chimeric NDV VLPs, wherein said VLPs NDV M protein and NDV F, FIN and/or NP protein, or portions thereof, and/or PIV F and/or FIN proteins, or portions thereof, and/or any chimeric protein described above.

As used herein, an “antibody” is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. Antibodies exist as intact immunoglobulins or as a number of well-characterized fragments produced by digestion with various peptidases.

In another embodiment, the invention comprises a method of inducing a protective cellular response to an infection or at least one symptom thereof in a subject, comprising administering at least one effective dose of chimeric NDV VLPs, wherein said VLP comprises NDV M protein and NDV F, HN and/or NP protein, or portions thereof, and/or PIV F and/or HN proteins, or portions thereof, and/or any chimeric protein described above. Cell-mediated immunity also plays a role in recovery from infection and may prevent additional complication and contribute to long term immunity.

As mentioned above, the invention of the VLPs prevent or reduce at least one symptom of an infection in a subject. Most symptoms of most infections are well known in the art. Thus, the method of the invention comprises the prevention or reduction of at least one symptom associated with an infection. A reduction in a symptom may be determined subjectively or objectively, e.g., self assessment by a subject, by a clinician's assessment or by conducting an appropriate assay or measurement (e.g. body temperature), including, e.g., a quality of life assessment, a slowed progression of a RSV infection or additional symptoms, a reduced severity of a RSV symptoms or a suitable assays (e.g. antibody titer and/or T-cell activation assay). The objective assessment comprises both animal and human assessments.

This invention is further illustrated by the following examples that should not be construed as limiting. The contents of all references, patents and published patent applications cited throughout this application, as well as the FIGURE and the Sequence Listing, are incorporated herein by reference for all purposes.

### EXAMPLES

### Example 1

### Chimeric NDV VLPS comprising Influenza Proteins

Constructs of membrane (M) and/or nucleoprotein (NP) proteins from New Castle Disease Virus and chimeric proteins comprising the external domains of influenza HA and/or NA protein sequences fused to the transmembrane and/or C-terminal domains of NDV HN and/or F are constructed (see SEQ ID NO 10 for an example). These constructs are illustrated in FIG. 1. The constructs are codon optimized and then cloned through a series of steps (as described above) into a bacmid vectors followed by rescue of recombinant baculovirus by plaque isolation. Insect cells are then infected and grown under conditions to allow VLP formation. The VLPs are isolated and purified as described above.

### Example 2

### Chimeric NDV VLPs

In order to form VLPs for other targets with the NDV core, native and/or chimeric molecules are cloned into a baculovirus. Chimeric VLP are made by expressing the M and NP genes from NDV and a chimeric protein comprising the transmembrane and C-terminal domain of NDV HN or F proteins fused to the external domains of proteins from infectious agents, such as VZV, RSV, Dengue virus. Such constructs are codon optimization and cloned through a series of steps (described above) into a bacmid followed by rescue of recombinant baculovirus by plaque isolation.

The VLPs for each of these targets are rescued by co-infection with the use of recombinant baculoviruses (1) expressing the NDV M and/or NP for VLP core formation and (2) expressing the chimeric proteins as described above. Below are representative NDV protein sequences that can be used in making chimeric NDV VLPs.

NDV M protein

(SEQ ID NO: 1)

MDSSRTIGLYFDSALPSSNLLAFPIVLRDVGDGKKQITPQYRIRRLDSWT
DSKEDSVFITTYGFIFQVGNEEVTVGMINDNPKRELLSAAMLCLGSVPNV
GDPVELARACLTMVVTCKKSATNTERMVFSVVQAPRVLQSCRVVADKYSS
VNAVKHVKAPEKIPGSETLEYKVNFVSLTVVPKKDVYKIPTAVLKVSGSS
LYNLALNVTIDVEVDPKSPLVKSLSRSDSGYYANLFLHIGLMSTVDKRGK
KVTFDQLERKIRRLDLSVGLSDVLGPSVLVKARGARTRLLAPFFSNSGTA
CYPIANASPQVAKILWSQTACLRSVKIIIQAGTQRAVAVTADHEVTSTKI
EKRHTIAKYNPFKK*
NDV F protein

(SEQ ID NO: 2)

MGPRSSTRIPVPLMLTIRITLALSYVRLTSSLDGRPLAAAGIVVTGDKAV
NIYTSSQTGSIIVKLLPNMPKDKEACAKAPLEAYNRTLTTLLTPLGDSIR
RIQESVTTSGGRRQRRFIGAIIGSVALGVATAAQITAASALIQANQNAAN
ILRLKESIAATNEAVHEVTGGLSQLAVAVGKMQQFVNDQFNNTAQELDCI
KIAQQVGVELNLYLTELTTVFGPQITSPALTQLTVQALYNLAGGNVDYLL
TKLGVGNNQLSSLIGSGLITGNPIFYDSQTQLLGIQVTLPSVGNLNNMRA
TYLETLSVSTTKGFASALVPKVVTQVGSVIEELDTSYCIEADLDLYCTRI
VTFPMSPGIYSCLSGNTSACMYSKTEGALTTPYMTLKGSVVANCQMTTCR
CADPPGIISQNYGEAVSLIDKHSCNVVSLDGITLRLSGEFDATYQKNISI
LDSQVLVTGNLDISTELGNVNHSISNALDKLEESNSKLDKVNVRLTSTSA
LITYIVLTVISLVLGMLSLVLACYLMYKQKAQRKTLLWLGNNTLDQMRAT
TKM*
Transmembrane Region Prediction for NDV_F

| | | |
|---|---|---|
| | Begin | End |
| Outside | 1 | 502 |
| Transmembrane Helix | 503 | 525 |
| Inside (cytoplasmic) | 526 | 553 |
| NDV HN protein | | |

(SEQID NO: 3)

MDSAVSQVALENDRREAKDTWRLVFRIAALLLMVITLAVSAVALAYSMEA
STPGDLVSIPTAIYRAEERITSALGSNQDVVDRIYKQVALESPLALLNTE
SIIMNAITSLSYQINGATNNSGCGAPVHDPDYIGGIGKELIVDDTSDVTS
FYPSAFQEHLNFIPAPTTGSGCTRIPSFDMSATHYCYTHNVILSGCRDHS
HSHQYLALGVLRTSATGRVFFSTLRSINLDDAQNRKSCSVSATPLGCDML
CSKITETEEEDYKSVIPTSMVHGRLGFDGQYHEKDLDVTTLFRDWVANYP
GVGGGSFINNRVWFPVYGGLKPSSPSDTAQEGRYVIYKRYNDTCPDEQDY
QIRMAKSSYKPGRFGGKRVQQAILSIKVSTSLGEDPVLTVPPNTVALMGA
EGRVLTVGTSHFLYQRGSSYFSPALLYPMTVNNKTATLHNPYTFNAFTRP
GSVPCQASARCPNSCVTGVYTDPYPLVFHRNHTLRGVFGTMLDDKQARLN
PVSAVFDNISRSRITRVSSSSTRAAYTTSTCFKVVKTNKTYCLSIAEISN
TLFGEFRIVPLLVEILKDGGV*
NDV NP protein

(SEQ ID NO: 4)

MSSVFDEYEQLLAAQTRPNGAHGGGEKGSTLKVEVPVFTLNSDDPEDRWN
FAVFCLRIAVSEDANKPLRQGALISLLCSHSQVMRNHVALAGKQNEATLA
VLEIDGFTNSVPQFNNRSGVSEERAQRFLMIAGSLPRACSNGTPFTTAGV
EDDAPEDITDTLERIISIQAQVWVTVAKAMTAYETADESETRRINKYMQQ
GRVQKKYILHPVCRSAIQLTIRQSLAVRIFLVSELKRGRNTAGGSSTYYS
LVGDIDSYIRNTGLTAFFLTLKYGINTKTSALALSSLAGDIQKMKQLMRL
YRMKGDNAPYMTLLGDSDQMSFAPAEYAQLYSFAMGMASVLDKGTGKYQF
ARDFMSTSFWRLGVEYARAQGSSINEDMAAELKLTPAARRGLAAAAQRAS
EETGSMDIPTQQAGVLTGLSDGGPQAPQGGLNRSQGQPDAGDGETQFLDL
MRAVANSMREAPNSVQNTTQQEPPSTPGPSQDNDTDWGY*
The following are examples of codon optimized (for
insect cells) nucleotide sequences for NDV
proteins
Codon optimized NDV M protein

(SEQ ID NO: 5)

ATGGACTCTTCTAGAACCATCGGTTTGTACTTCGACTCTGCTTTGCCATC
TTCTAACTTGTTGGCTTTCCCAATCGTTTTGAGAGACGTTGGTGACGGTA
AGAAGCAAATCACCCCACAATACAGAATCAGAAGATTGGACTCTTGGACC
GACTCTAAGGAAGACTCTGTTTTCATCACCACCTACGGTTTCATCTTCCA
AGTTGGTAACGAAGAAGTTACCGTTGGTATGATCAACGACAACCCAAAGA
GAGAATTGTTGTCTGCTGCTATGTTGTGTTTGGGTTCTGTTCCAAACGTT
GGTGACCCAGTTGAATTGGCTAGAGCTTGTTTGACCATGGTTGTTACCTG
TAAGAAGTCTGCTACCAACACCGAAAGAATGGTTTTCTCTGTTGTTCAAG
CTCCAAGAGTTTTGCAATCTTGTAGAGTTGTTGCTGACAAGTACTCTTCT
GTTAACGCTGTTAAGCACGTTAAGGCTCCAGAAAAGATCCCAGGTTCTGA
AACCTTGGAATACAAGGTTAACTTCGTTTCTTTGACCGTTGTTCCAAAGA
AGGACGTTTACAAGATCCCAACCGCTGTTTTGAAGGTTTCTGGTTCTTCT
TTGTACAACTTGGCTTTGAACGTTACCATCGACGTTGAAGTTGACCCAAA
GTCTCCATTGGTTAAGTCTTTGTCTAGATCTGACTCTGGTTACTACGCTA
ACTTGTTCTTGCACATCGGTTTGATGTCTACCGTTGACAAGAGAGGTAAG
AAGGTTACCTTCGACCAATTGGAAAGAAAGATCAGAAGATTGGACTTGTC
TGTTGGTTTGTCTGACGTTTTGGGTCCATCTGTTTTGGTTAAGGCTAGAG
GTGCTAGAACCAGATTGTTGGCTCCATTCTTCTCTAACTCTGGTACCGCT
TGTTACCCAATCGCTAACGCTTCTCCACAAGTTGCTAAGATCTTGTGGTC
TCAAACCGCTTGTTTGAGATCTGTTAAGATCATCATCCAAGCTGGTACCC
AAAGAGCTGTTGCTGTTACCGCTGACCACGAAGTTACCTCTACCAAGATC
GAAAAGAGACACACCATCGCTAAGTACAACCCATTCAAGAAGTGA
Codon optimized NDV F protein

(SEQ ID NO: 6)

ATGGGTCCAAGATCTTCTACCAGAATCCCAGTTCCATTGATGTTGACCAT
CAGAATCACCTTGGCTTTGTCTTACGTTAGATTGACCTCTTCTTTGGACG
GTAGACCATTGGCTGCTGCTGGTATCGTTGTTACCGGTGACAAGGCTGTT
AACATCTACACCTCTTCTCAAACCGGTTCTATCATCGTTAAGTTGTTGCC
AAACATGCCAAAGGACAAGGAAGCTTGTGCTAAGGCTCCATTGGAAGCTT
ACAACAGAACCTTGACCACCTTGTTGACCCCATTGGGTGACTCTATCAGA
AGAATCCAAGAATCTGTTACCACCTCTGGTGGTAGAAGACAAAGAAGATT
CATCGGTGCTATCATCGGTTCTGTTGCTTTGGGTGTTGCTACCGCTGCTC
AAATCACCGCTGCTTCTGCTTTGATCCAAGCTAACCAAAACGCTGCTAAC
ATCTTGAGATTGAAGGAATCTATCGCTGCTACCAACGAAGCTGTTCACGA
AGTTACCGGTGGTTTGTCTCAATTGGCTGTTGCTGTTGGTAAGATGCAAC
AATTCGTTAACGACCAATTCAACAACACCGCTCAAGAATTGGACTGTATC
AAGATCGCTCAACAAGTTGGTGTTGAATTGAACTTGTACTTGACCGAATT
GACCACCGTTTTCGGTCCACAAATCACCTCTCCAGCTTTGACCCAATTGA
CCGTTCAAGCTTTGTACAACTTGGCTGGTGGTAACGTTGACTACTTGTTG
ACCAAGTTGGGTGTTGGTAACAACCAATTGTCTTCTTTGATCGGTTCTGG
TTTGATCACCGGTAACCCAATCTTCTACGACTCTCAAACCCAATTGTTGG
GTATCCAAGTTACCTTGCCATCTGTTGGTAACTTGAACAACATGAGAGCT
ACCTACTTGGAAACCTTGTCTGTTTCTACCACCAAGGGTTTCGCTTCTGC
TTTGGTTCCAAAGGTTGTTACCCAAGTTGGTTCTGTTATCGAAGAATTGG
ACACCTCTTACTGTATCGAAGCTGACTTGGACTTGTACTGTACCAGAATC
GTTACCTTCCCAATGTCTCCAGGTATCTACTCTTGTTTGTCTGGTAACAC
CTCTGCTTGTATGTACTCTAAGACCGAAGGTGCTTTGACCACCCCATACA
TGACCTTGAAGGGTTCTGTTGTTGCTAACTGTCAAATGACCACCTGTAGA
TGTGCTGACCCACCAGGTATCATCTCTCAAAACTACGGTGAAGCTGTTTC
TTTGATCGACAAGCACTCTTGTAACGTTGTTTCTTTGGACGGTATCACCT
TGAGATTGTCTGGTGAATTCGACGCTACCTACCAAAAGAACATCTCTATC
TTGGACTCTCAAGTTTTGGTTACCGGTAACTTGGACATCTCTACCGAATT
GGGTAACGTTAACCACTCTATCTCTAACGCTTTGGACAAGTTGGAAGAAT
CTAACTCTAAGTTGGACAAGGTTAACGTTAGATTGACCTCTACCTCTGCT
TTGATCACCTACATCGTTTTGACCGTTATCTCTTTGGTTTTGGGTATGTT
GTCTTTGGTTTTGGCTTGTTACTTGATGTACAAGCAAAAGGCTCAAAGAA
AGACCTTGTTGTGGTTGGGTAACAACACCTTGGACCAAATGAGAGCTACC
ACCAAGATGTGA
Codon optimized NDV HN protein

(SEQ ID NO: 7)

ATGGACTCTGCTGTTTCTCAAGTTGCTTTGGAAAACGACAGAAGAGAAGC
TAAGGACACCTGGAGATTGGTTTTCAGAATCGCTGCTTTGTTGTTGATGG
TTATCACCTTGGCTGTTTCTGCTGTTGCTTTGGCTTACTCTATGGAAGCT
TCTACCCCAGGTGACTTGGTTTCTATCCCAACCGCTATCTACAGAGCTGA
AGAAAGAATCACCTCTGCTTTGGGTTCTAACCAAGACGTTGTTGACAGAA
TCTACAAGCAAGTTGCTTTGGAATCTCCATTGGCTTTGTTGAACACCGAA
TCTATCATCATGAACGCTATCACCTCTTTGTCTTACCAAATCAACGGTGC
TACCAACAACTCTGGTTGTGGTGCTCCAGTTCACGACCCAGACTACATCG
GTGGTATCGGTAAGGAATTGATCGTTGACGACACCTCTGACGTTACCTCT
TTCTACCCATCTGCTTTCCAAGAACACTTGAACTTCATCCCAGCTCCAAC
CACCGGTTCTGGTTGTACCAGAATCCCATCTTTCGACATGTCTGCTACCC
ACTACTGTTACACCCACAACGTTATCTTGTCTGGTTGTAGAGACCACTCT
CACTCTCACCAATACTTGGCTTTGGGTGTTTTGAGAACCTCTGCTACCGG
TAGAGTTTTCTTCTCTACCTTGAGATCTATCAACTTGGACGACGCTCAAA
ACAGAAAGTCTTGTTCTGTTTCTGCTACCCCATTGGGTTGTGACATGTTG
TGTTCTAAGATCACCGAAACCGAAGAAGAAGACTACAAGTCTGTTATCCC
AACCTCTATGGTTCACGGTAGATTGGGTTTCGACGGTCAATACCACGAAA
AGGACTTGGACGTTACCACCTTGTTCAGAGACTGGGTTGCTAACTACCCA
GGTGTTGGTGGTGGTTCTTTCATCAACAACAGAGTTTGGTTCCCAGTTTA
CGGTGGTTTGAAGCCATCTTCTCCATCTGACACCGCTCAAGAAGGTAGAT
ACGTTATCTACAAGAGATACAACGACACCTGTCCAGACGAACAAGACTAC
CAAATCAGAATGGCTAAGTCTTCTTACAAGCCAGGTAGATTCGGTGGTAA
GAGAGTTCAACAAGCTATCTTGTCTATCAAGGTTTCTACCTCTTTGGGTG
AAGACCCAGTTTTGACCGTTCCACCAAACACCGTTGCTTTGATGGGTGCT
GAAGGTAGAGTTTTGACCGTTGGTACCTCTCACTTCTTGTACCAAAGAGG
TTCTTCTTACTTCTCTCCAGCTTTGTTGTACCCAATGACCGTTAACAACA
AGACCGCTACCTTGCACAACCCATACACCTTCAACGCTTTCACCAGACCA
GGTTCTGTTCCATGTCAAGCTTCTGCTAGATGTCCAAACTCTTGTGTTAC
CGGTGTTTACACCGACCCATACCCATTGGTTTTCCACAGAAACCACACCT
TGAGAGGTGTTTTCGGTACCATGTTGGACGACAAGCAAGCTAGATTGAAC
CCAGTTTCTGCTGTTTTCGACAACATCTCTAGATCTAGAATCACCAGAGT
TTCTTCTTCTTCTACCAGAGCTGCTTACACCACCTCTACCTGTTTCAAGG
TTGTTAAGACCAACAAGACCTACTGTTTGTCTATCGCTGAAATCTCTAAC
ACCTTGTTCGGTGAATTCAGAATCGTTCCATTGTTGGTTGAAATCTTGAA
GGACGGTGGTGTTTGA
Codon optimized NDV NP protein

(SEQ ID NO: 8)

ATGTCTTCTGTTTTCGACGAATACGAACAATTGTTGGCTGCTCAAACCAG
ACCAAACGGTGCTCACGGTGGTGGTGAAAAGGGTTCTACCTTGAAGGTTG
AAGTTCCAGTTTTCACCTTGAACTCTGACGACCCAGAAGACAGATGGAAC
TTCGCTGTTTTCTGTTTGAGAATCGCTGTTTCTGAAGACGCTAACAAGCC
ATTGAGACAAGGTGCTTTGATCTCTTTGTTGTGTTCTCACTCTCAAGTTA
TGAGAAACCACGTTGCTTTGGCTGGTAAGCAAAACGAAGCTACCTTGGCT
GTTTTGGAAATCGACGGTTTCACCAACTCTGTTCCACAATTCAACAACAG
ATCTGGTGTTTCTGAAGAAAGAGCTCAAAGATTCTTGATGATCGCTGGTT
CTTTGCCAAGAGCTTGTTCTAACGGTACCCCATTCACCACCGCTGGTGTT
GAAGACGACGCTCCAGAAGACATCACCGACACCTTGGAAAGAATCATCTC
TATCCAAGCTCAAGTTTGGGTTACCGTTGCTAAGGCTATGACCGCTTACG
AAACCGCTGACGAATCTGAAACCAGAAGAATCAACAAGTACATGCAACAA
GGTAGAGTTCAAAAGAAGTACATCTTGCACCCAGTTTGTAGATCTGCTAT
CCAATTGACCATCAGACAATCTTTGGCTGTTAGAATCTTCTTGGTTTCTG
AATTGAAGAGAGGTAGAAACACCGCTGGTGGTTCTTCTACCTACTACTCT
TTGGTTGGTGACATCGACTCTTACATCAGAAACACCGGTTTGACCGCTTT
CTTCTTGACCTTGAAGTACGGTATCAACACCAAGACCTCTGCTTTGGCTT
TGTCTTCTTTGGCTGGTGACATCCAAAAGATGAAGCAATTGATGAGATTG
TACAGAATGAAGGGTGACAACGCTCCATACATGACCTTGTTGGGTGACTC
TGACCAAATGTCTTTCGCTCCAGCTGAATACGCTCAATTGTACTCTTTCG
CTATGGGTATGGCTTCTGTTTTGGACAAGGGTACCGGTAAGTACCAATTC
GCTAGAGACTTCATGTCTACCTCTTTCTGGAGATTGGGTGTTGAATACGC
TAGAGCTCAAGGTTCTTCTATCAACGAAGACATGGCTGCTGAATTGAAGT
TGACCCCAGCTGCTAGAAGAGGTTTGGCTGCTGCTGCTCAAAGAGCTTCT
GAAGAAACCGGTTCTATGGACATCCCAACCCAACAAGCTGGTGTTTTGAC
CGGTTTGTCTGACGGTGGTCCACAAGCTCCACAAGGTGGTTTGAACAGAT
CTCAAGGTCAACCAGACGCTGGTGACGGTGAAACCCAATTCTTGGACTTG
ATGAGAGCTGTTGCTAACTCTATGAGAGAAGCTCCAAACTCTGTTCAAAA
CACCACCCAACAAGAACCACCATCTACCCCAGGTCCATCTCAAGACAACG
ACACCGACTGGGGTTACTGA
Influenza HA sequence

(SEQ ID NO 9)

MKTIIALSYI LCLVFAQKLP GNDNSTATLC LGHHAVPNGT
IVKTITNDQI EVTNATELVQ SSSTGGICDS PHQILDGENC
TLIDALLGDP QCDGFQNKKW DLFVERSKAY SNCYPYDVPD
YASLRSLVAS SGTLEFNNES FNWTGVTQNG TSSACKRRSN
KSFFSRLNWL THLKYKYPAL NVTMPNNEKF DKLYIWGVLH
PGTDSDQISL YAQASGRITV STKRSQQTVI PNIGSRPRVR
DVSSRISIYW TIVKPGDILL INSTGNLIAP RGYFKIRSGK
SSIMRSDAPI GKCNSECITP NGSIPNDKPF QNVNRITYGA
CPRYIKQNTL KLATGMRNVP EKQTRGIFGA IAGFIENGWE
GMVDGWYGFR HQNSEGTGQA ADLKSTQAAI NQINGKLNRL
IGKTNEKFHQ IEKEFSEVEG RIQDLEKYVE DTKIDLWSYN
AELLVALENQ HTIDLTDSEM NKLFERTKKQ LRENAEDMGN
GCFKIYHKCD NACIGSIRNG TYDHDVYRDE ALNNRFQIKG
VELKSGYKDW ILWISFAISC FLLCVALLGF IMWACQKGNI
RCNICI*
Transmembrane Region Prediction for
Influenza A H3N2 (Fujian strain) HA protein

| | | |
|---|---|---|
| | Begin | End |
| Outside | 1 | 529 |
| TM Helix | 530 | 552 |
| Inside (cytoplasmic) | 553 | 566 |

Proposed chimeric [Influenza HA]-[NDV F] protein

(SEQ ID NO 10)

MKTIIALSYI LCLVFAQKLP GNDNSTATLC LGHHAVPNGT
IVKTITNDQI EVTNATELVQ SSSTGGICDS PHQILDGENC
TLIDALLGDP QCDGFQNKKW DLFVERSKAY SNCYPYDVPD
YASLRSLVAS SGTLEFNNES FNWTGVTQNG TSSACKRRSN
KSFFSRLNWL THLKYKYPAL NVTMPNNEKF DKLYIWGVLH
PGTDSDQISL YAQASGRITV STKRSQQTVI PNIGSRPRVR
DVSSRISIYW TIVKPGDILL INSTGNLIAP RGYFKIRSGK
SSIMRSDAPI GKCNSECITP NGSIPNDKPF QNVNRITYGA
CPRYIKQNTL KLATGMRNVP EKQTRGIFGA IAGFIENGWE
GMVDGWYGFR HQNSEGTGQA ADLKSTQAAI NQINGKLNRL
IGKTNEKFHQ IEKEFSEVEG RIQDLEKYVE DTKIDLWSYN
AELLVALENQ HTIDLTDSEM NKLFERTKKQ LRENAEDMGN
GCFKIYHKCD NACIGSIRNG TYDHDVYRDE ALNNRFQIKG
VELKSGYKDT YIVLTVISLV LGMLSLVLAC YLMYKQKAQR
KTLLWLGNNT LDQMRATTKM
Amino acid residues

| | |
|---|---|
| 1-529 | Fujian HA |
| 530-580 | NDV F(Transmembrane helix double under- |
| | lined and cytoplasmic domain underlined) |

All publications and patent applications herein are incorporated by reference to the same extent as if each individual publication or patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A chimeric virus like particle (VLP) comprising a Newcastle Disease Virus (NDV) core protein (M) and at least one protein from a different infectious agent, wherein the VLP is noninfectious and does not comprise genetic material encoding for the proteins; and wherein the at least one protein is fused to a NDV protein or fragment thereof that associates with the VLP.

2. The VLP of claim 1, wherein said protein from a different infectious agent is a viral protein.

3. The VLP of claim 2, wherein said viral protein is an envelope associated protein.

4. The VLP of claim 3, wherein said envelope associated protein is expressed on the surface of the VLP.

5. The VLP of claim 3, wherein said envelope associated protein comprises an epitope that will generate a protective immune response in a vertebrate.

6.-91. (canceled)

92. The VLP of claim 1, wherein said NDV protein or fragment thereof is selected from the group consisting of NP, F, HN, the transmembrane and/or C terminal end of NP, the transmembrane and/or C terminal end of F, and the transmembrane and/or C terminal end of HN.

93. The VLP of claim 1, wherein said NDV protein or fragment thereof associates with the NDV M protein.

94. The VLP of claim 2, wherein said viral protein is from a virus selected the group consisting of influenza virus, dengue virus, yellow virus, Herpes simplex virus I and II, rabies virus, parainfluenza virus, varicella zoster virus, respiratory syncytial virus, rabies virus, human immunodeficiency virus, corona virus, and hepatitis virus.

95. The VLP of claim 94, wherein said virus is influenza virus and the viral protein is HA and/or NA.

96. The VLP of claim 94, wherein said virus is respiratory syncytial virus and the viral protein is F and/or G.

97. A method of producing a chimeric VLP of claim 1, comprising transfecting at least one vector encoding a Newcastle Disease Virus (NDV) viral core protein (M) and at least one protein from a different infectious agent into a cell and expressing said vectors under conditions that allow VLPs to be formed.

98. The method of claim 97, wherein said NDV protein or fragment thereof is selected from the group consisting of NP, F, and HN proteins.

99. The method of claim 97, wherein the at least one protein from a different infectious agent is the influenza virus viral protein HA and/or NA.

100. The method of claim 97, wherein the at least one protein from a different infectious agent is the respiratory syncytial virus viral protein F and/or G.

101. An antigenic formulation comprising a chimeric VLP of claim 1.

102. A vaccine comprising a chimeric VLP of claim 1.

103. A method of inducing an immune response in a vertebrate comprising administering to said vertebrate chimeric VLPs comprising a Newcastle Disease Virus (NDV) viral core protein (M) and at least one protein from a different infectious agent, wherein the VLP is noninfectious and does not comprise genetic material encoding for the proteins; and wherein the at least one protein is fused to a NDV protein or fragment thereof that associates with the VLP.

104. The method of claim 103, wherein said immune response is a humoral immune response.

105. The method of claim 103, wherein said immune response is a cellular immune response.

106. The method of claim 103, wherein said at least one protein from a different infectious agent is a respiratory syncytial virus F and/or G protein.
